(19) United States
(12) Patent Application Publication
(10) Pub. No.: US 2015/0190249 A1
(43) Pub. Date: Jul. 9, 2015

(54) Wearable Power Assist System

(71) Applicants: Masayoshi Ishibashi, Tokyo (JP); Midori Kato, Tokyo (JP)

(72) Inventors: Masayoshi Ishibashi, Tokyo (JP); Midori Kato, Tokyo (JP)

(73) Assignee: Hitachi, Ltd., Chiyoda-ku, Tokyo (JP)

(21) Appl. No.: 14/410,766

(22) PCT Filed: Jun. 27, 2012
(86) PCT No.: PCT/JP2012/066320

§ 371 Date: Dec. 23, 2014

(51) Int. Cl. A61F 2/70; G05B 15/02

(52) U.S. Cl. CPC A61F 2/70; G05B 15/02; A61F 2002/701

(57) ABSTRACT

An object of the disclosed invention is to provide a wearable power assist system that is light weight and capable of assisting in various situations. The invention is used to select a joint to be assisted according to a desired action and transmitting a force generated by an actuator to assist the joint and to an assist the outer skeleton.

### TECHNICAL FIELD

The present invention relates to a wearable power assist system, and for example, to a configuration of and control method for the same.

### BACKGROUND ART

Population aging is progressing globally. With this, various problems such as welfare measures for the elderly, nursing care issues and labor issues are becoming more visible. To solve such problems, robots for assisting human functions are drawing attention. Of such robots, a wearable power assist system which is worn to assist lower limb functions and upper limb functions is considered useful for rehabilitation, assistance for independence of a person requiring nursing care, assistance for a caregiver, and walking assistance in order to prevent the need of nursing care.

### SUMMARY OF INVENTION

### Technical Problem

Human actions such as nursing care and walking are often done, moving a plurality of joints at a time. If all of those joints are to be assisted separately, it requires the same number of actuators as the joints. If the number of actuators increases, not only the total weight of the actuators themselves increases but also the power source to move the actuators increases in size, causing the system to become large and heavy. If the system becomes large, it is difficult to use the system for walking assistance or the like when going out, or to use the system for the purpose of leading everyday life while wearing the system.

Also, some rehabilitation programs use a light-weight system because these programs can be dealt with by assisting a specific joint only. However, the use is limited.

### Solution to Problem

In order to solve such problems, in a wearable power assist system according to the invention, a joint to be assisted is selected according to an action, and a force generated by an actuator to assist the joint is transmitted to an assist outer skeleton. That is, a force generated by a single actuating device is shifted to and used on a required assist outer skeleton. By doing so, it is possible to assist various actions even with a small number of actuators.

The above measure is based on the knowledge that, while different forces are applied to joints in various walking states, an assistance effect is achieved simply by assisting one joint that plays an important role in each walking state.

For example, typical stair ascent is an action as follows. First, a person places the body weight on one leg (here, the right leg) and lifts up the other leg (left leg) to place the left leg on the step that is immediately above. At this point, the hip joint and the knee joint of the left leg are bent. Next, while shifting the body weight to the left foot simultaneously with kicking the floor via the right angle joint, the person extends the hip joint and the knee joint of the left leg, thus moving the body one step above. That is, at the time of stair ascent, a force is applied to all of the ankle joint, the hip joint and the knee joint. However, as a result of an experiment, it is found that many able-bodied people feel an assistance effect simply by having assistance with the knee joint to extend at the timing of extending the knee joint. Similarly, in another walking state, while a force is applied to a plurality of joints, a result showing that an assistance effect is achieved simply by assisting a pair of joints that plays an important role, is obtained.

### Advantageous Effect of Invention

According to the invention, a wearable power assist system which is light-weight and capable of assisting in various situations is provided.

### BRIEF DESCRIPTION OF DRAWINGS

FIG. 1 is a schematic view for explaining the configuration of the invention in the case of assisting lower limbs.

FIG. 2 is a schematic view for explaining the outline of a wearable power assist system of the invention. FIG. 2(a) is a schematic view showing the use at the time of stair ascent. FIG. 2(b) is a schematic view showing the use at the time of flatland walk.

FIG. 3 is a schematic view of an actuating device.

FIG. 4 is a schematic structural view of an assist outer skeleton in the state where a drive force transmission unit is not attached thereto. FIG. 4(a) is a schematic view seen from the front. FIG. 4(b) is a schematic view seen from the side. FIG. 4(c) is a schematic view seen from obliquely above.

FIG. 5 is a schematic structural view of the assist outer skeleton in state where a drive force transmission unit is attached thereto. FIG. 5(a) is a schematic view seen from the front. FIG. 5(b) is a schematic view seen from the side.

FIG. 6 is a schematic view showing a movement of the assist outer skeleton. FIG. 6(a) shows the state where the wire is loosened. FIG. 6(b) shows the state where the wire is pulled to force extension.

FIG. 7 is a schematic view showing a movement of the assist outer skeleton with a center wire guide attached near the rotating shaft. FIG. 7(a) shows the state where the wire is loosened. FIG. 7(b) shows the state where the wire is pulled to force extension.

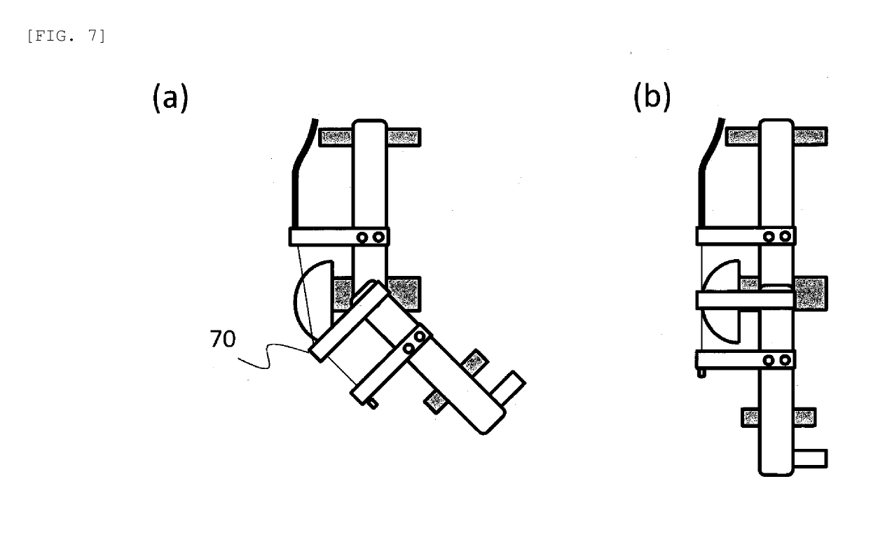

FIG. 8 is a schematic view showing a movement of the assist outer skeleton. FIG. 8(a) shows an extended state. FIG. 8(b shows a bent state.

FIG. 9 is a schematic view of an actuating device.

FIG. 10 is a schematic structural view of a measuring insole.

FIG. 11 shows a basic control flow of the wearable power assist system.

FIG. 12 shows a typical example of change with time in the knee joint angle of the right leg, knee angular velocity, and the pressure applied to the big toe, at the time of stair ascent.

FIG. 13 is a schematic view of a knee joint assist outer skeleton which will not cause much rubbing.

FIG. 14 is a schematic view for explaining the configuration of a wearable power assist system.

FIG. 15 is a schematic view for explaining actuators and switching devices of a selective actuating device. FIG. 15(a) is a schematic view seen from the top. FIG. 15(b) is a schematic view seen from the side when a select tension pulley is not pressed. FIG. 15(c) is a schematic view seen from the side when a select tension pulley is pressed.

FIG. 16 shows a typical example of change with time in the angle of each joint in flatland walk, stair ascent, and stair descent.

### DESCRIPTION OF EMBODIMENTS

### EXAMPLE 1

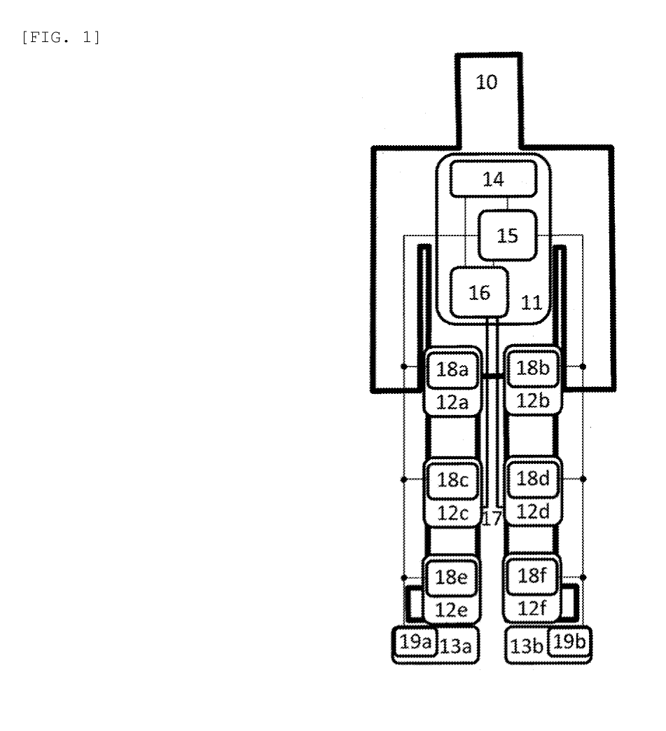

In Example 1, the configuration and operation of an embodiment of the wearable power assist system according to the invention will be described. FIG. 1 is a schematic view for explaining the configuration of the invention in the case of assisting lower limbs. A user 10 wears an actuation-control device housing unit 11 on the back, an assist outer skeleton 12 (right hip joint assist outer skeleton 12a, left hip joint assist outer skeleton 12b, right knee joint assist outer skeleton 12c, left knee joint assist outer skeleton 12d, right ankle joint assist outer skeleton 12e, left ankle joint assist outer skeleton 12f) on each joint, and a measuring insole 13 (right foot measuring insole 13a, left foot measuring insole 13b) for measuring sole pressure distribution, on the feet. A power source 14, an operation control unit 15, and an actuating device 16 are housed in the actuation-control device housing unit 11. The operation control unit 15 and the actuating device 16 are supplied with electricity from the power source 14. The respective assist outer skeletons 12a to 12f include angle sensors 18a to 18f for measuring the respective joint angles. The angle sensors 18a to 18f are connected to the operation control unit 15. The respective measuring insoles 13 include foot pressure measuring sensors 19a, 19b. The foot pressure measuring sensors 19a, 19b are connected to the operation control unit 15. The actuating device 16 is configured with two actuators in total for the right leg and the left leg, and a drive force transmission unit 17 is joined thereto so as to transmit a force generated by the actuating device 16 to the assist outer skeleton 12. While FIG. 1 shows an example in which the drive force transmission unit 17 is joined to the left and right knee joint assist outer skeletons, the drive force transmission unit 17 can be relocated to arbitrary ones of the assist outer skeletons 12a to 12f according to the need for assistance.

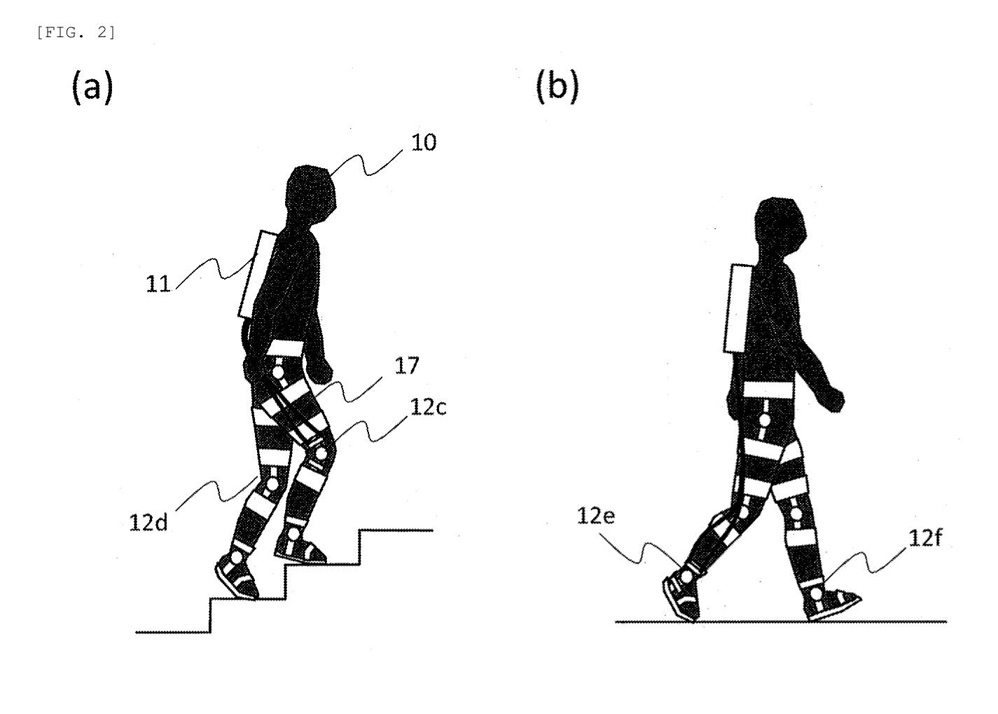

FIG. 2 is a schematic view for explaining the outline of the wearable power assist system according to the invention. FIG. 2a shows the use at the time of stair ascent. FIG. 2b shows the use at the time of flatland walk. At the time of stair ascent, the act of lifting up the body to the step above is assisted. Here, the extension of the knee joints when going up stairs is addicted by the left and right knee joint assist outer skeletons 12c, 12d. That is, the drive force transmission unit 17 is joined to the knee joint assist outer skeletons 12c, 12d, and operates the knee joint assist outer skeletons to extend with the timing when the knee joints are extended to lift up the body. Meanwhile, at the time of flatland walk, the act of kicking the ground is assisted by the left and right ankle joint assist outer skeletons 12e, 12f. Specifically, the drive force transmission unit 17 is jointed to the ankle joint assist outer skeletons 12e, 12f, and the ankle joint assist outer skeletons perform assistance such that the insteps extend with the timing when the ground is kicked via the ankles. By thus relocating the drive force transmission unit to joint assist outer skeletons that need assistance according to the walking state, it is possible to assist various walking states with only a set of actuators.

In our experiment, many people find assistance effective for the ankle joints in flatland walk and slope or stair descent and for the knee joints in slope or stair ascent. However, walking actions and target actions to be assisted vary from person to person. Therefore, some people may find it effective for the hip joints in flatland walk and for the knee joints in slope or stair descent. It is effective to set an assisting method suitable for each person in advance, and based on that, relocate the drive force transmission unit to the assist outer skeleton for each joint to give assistance according to the walking state.

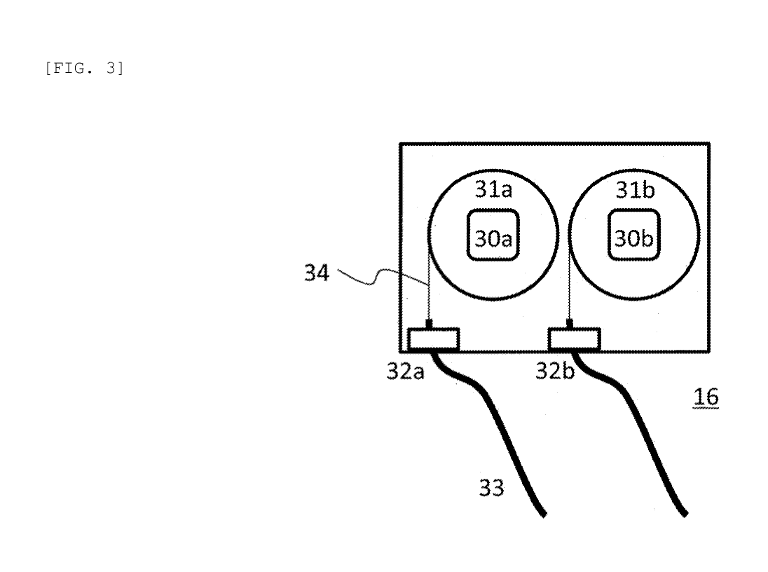

FIG. 3 shows a schematic view of the actuating device. In the actuating device 16, two motors 30a, 30b are fixed as actuators for the left and right legs, and the rotating shafts thereof are fixed to left and right pulleys 31a, 31b. The left and right pulleys 31a, 31b are jointed to wires 34 of the drive force transmission unit 17. The drive force transmission unit has a structure in which the wires 34 are inserted in housing pipes 33 that can be bent flexibly like brake wires of a bicycle. The wires 34 can move inside the housing pipes 33. The housing pipes 33 are fixed to the actuating device 16 at wire fixing sections 32a, 32b. By rotating the motors 30a, 30b, the actuating device 16 can pull or loosen the wires 34 of the drive force transmission unit 17. While FIG. 3 shows the configuration in which motors generating rotating motion are used as actuators, the wires 34 can be directly moved by a linear motion-type actuator such as an air pressure cylinder, hydraulic cylinder, or linear motor.

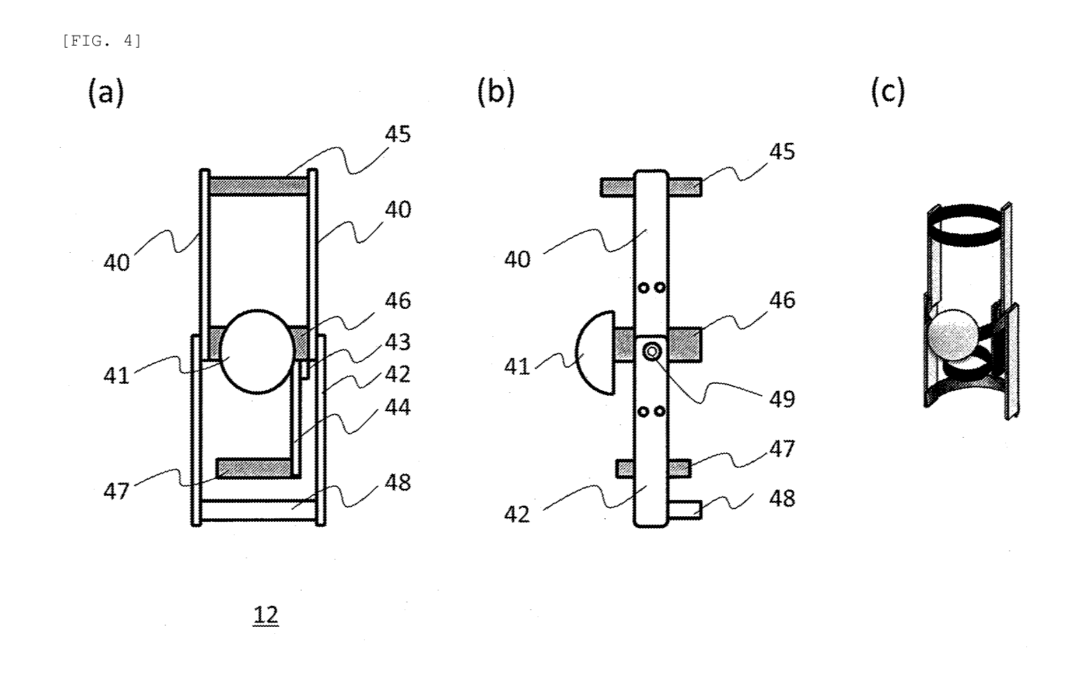

FIG. 4 is a schematic structural view of an assist outer skeleton in the state where the drive force transmission unit 17 is not attached thereto. Here, detailed explanation is given using the knee assist outer skeleton as an example. However, the basic configuration is the same for the hip and ankle. FIG. 4a is a schematic view seen from the front. FIG. 4b is a schematic view seen from the side. FIG. 4c is a schematic view seen from obliquely above. The configuration includes a fixed stay 40, a knee pad 41, a power stay 42, an angle sensor 43, a joint angle measuring stay 44, a thigh fixing belt 45, a knee fixing belt 46, a calf fixing belt 47, and a power belt 48. The joint angle measuring stay 44 and the power stay 42 are joined to the fixed stay 40 on a rotation center shaft 49 and form a link structure. The fixed stay 40, the knee pad 41, and the joint angle measuring stay 44 are fixed to the user's thigh, knee, and calf, respectively, with the thigh fixing belt 45, the knee fixing belt 46, and the calf fixing belt 47.

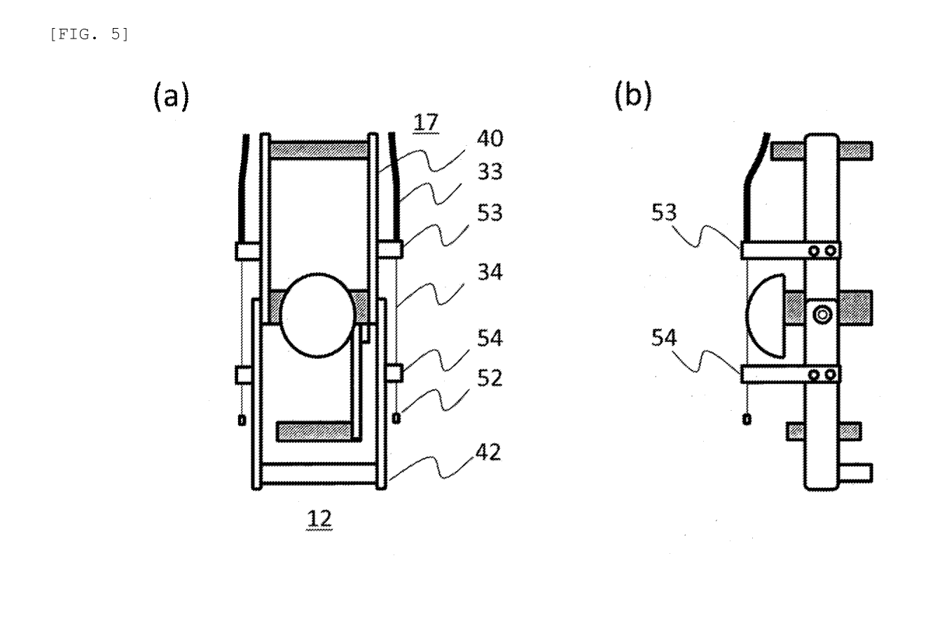

FIG. 5 is a schematic structural view of the assist outer skeleton in state where a drive force transmission unit 17 is attached thereto. FIG. 5(a) is a schematic view seen from the front. FIG. 5(b) is a schematic view seen from the side. The distal end of the housing pipe 33 is joined to an upper wire guide 53. The wire 34 penetrates a hole in a lower wire guide 54. A stopper 52 is provided at the distal end of the wire 34 so that the wire 34 will not come off of the hole in the lower wire guide 54. The upper wire guide 53 is joined to the fixed stay 40 and the lower wire guide 54 is joined to the power stay 42, by a method that allows easy detachment, such as a screw. While FIG. 5 shows an example in which the drive force transmission unit 17 is attached on both the outer side and the inner side of the knee, the drive transmission unit may be attached on the outer side alone or on the inner side alone according to need.

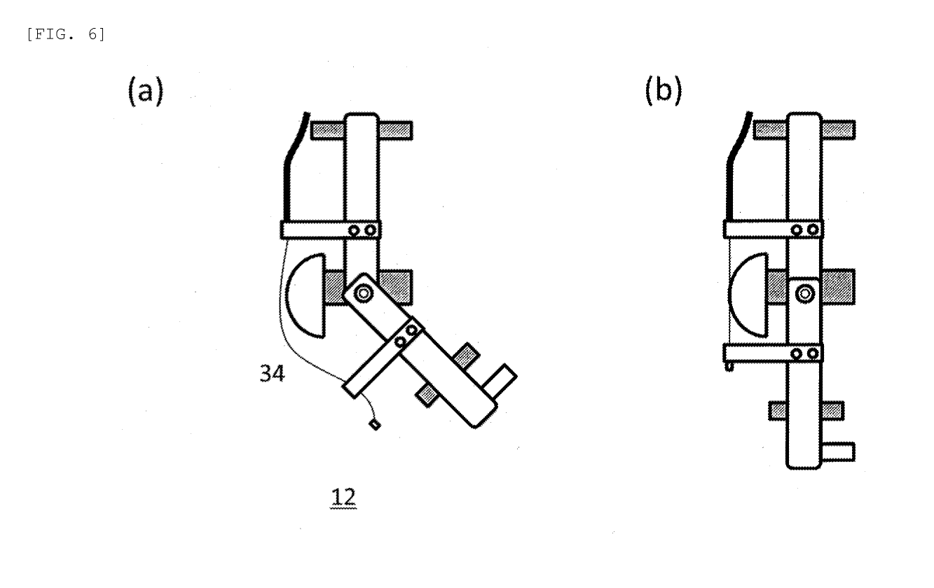

FIG. 6 is a schematic view showing a movement of the assist outer skeleton 12. In the state where the wire 34 is loosened as shown in FIG. 6a, the assist outer skeleton 12 can be in both an extended state and a bent state. However, the assist outer skeleton 12 can be forced into an extended state by pulling the wire 34 as shown in FIG. 6b. When the assist outer skeleton 12 in the state of being worn by the user is shifted from a bent state to an extended state, the user's leg is supported at the three points of the thigh fixing belt, the knee fixing belt, and the power belt, and the user feels a force such that the knee shifts from a bent state to an extended state.

The rotational torque of the assist outer skeleton is decided by the tensile force of the wire 34 and the length of the wire guide. Therefore, if a center wire guide 70 is provided near the rotating shaft so that the distance between the wire 34 and the rotating shaft becomes long when the assist outer skeleton is bent, a large rotational torque can be obtained with a small tensile force.

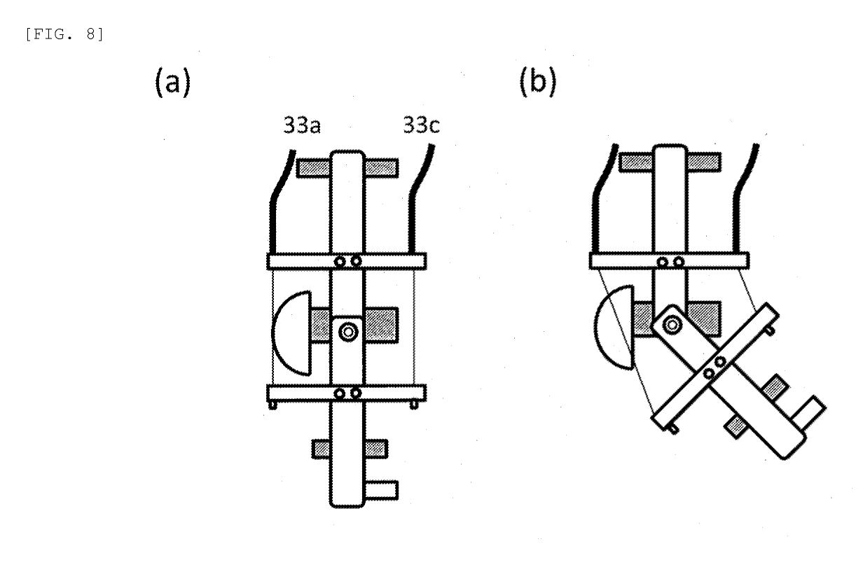

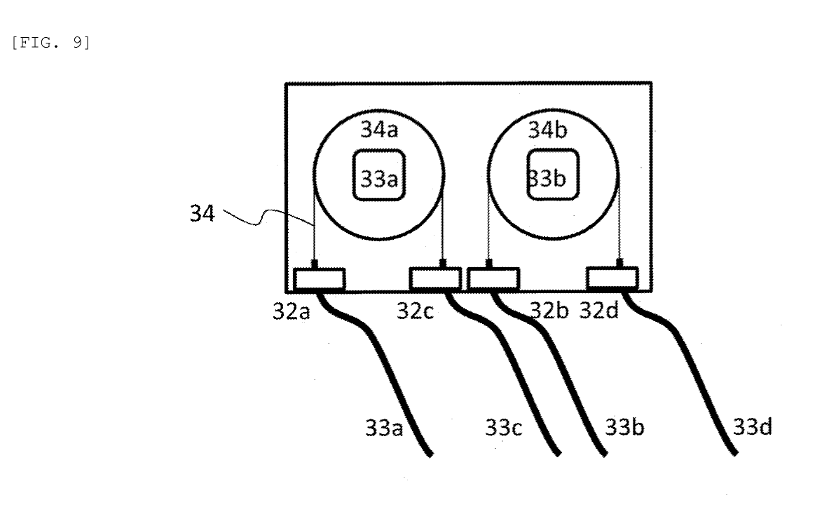

Since the assistance with the knee joint is often assistance in shifting from a bent state to an extended state, the structure explained with reference to FIG. 5 can cope in most cases. Moreover, in the case of coping with forcing a bent state from an extended state, a structure in which wires are attached on both sides of the rotating shaft as shown in FIG. 8 is employed. In this case, by attaching wires on both sides of the pulley of the actuating device 16 as shown in FIG. 9, fixing the wires to the actuating device 16 at wire fixing sections 32a, 32b, 32c, 32d, and joining a housing pipe 33a connected to the wire fixing section 32a, ahead of the rotating shaft, and a housing pipe 33c behind the rotating shaft, as shown in FIG. 8, it is possible to cope with assistance with both extension and bending without increasing the number of actuators.

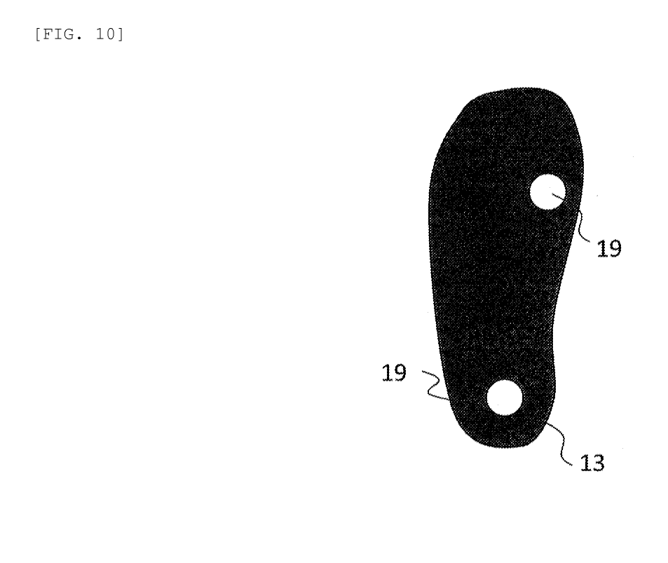

FIG. 10 shows the structure of a measuring insole for measuring sole pressure distribution. A structure in which a pressure sensor is attached to the surface of the insole 13 is placed under the foot and used as an insole of a shoe or sandal. If a pressure sensor is installed at two positions in total near the big toe and near the heel as shown in FIG. 10, sole pressure distribution can be measured and a proper assist timing can be decided. However, it is possible to use only one position near the big toe in order to simplify the system. Also, if a pressure sensor matrix is used, though the system becomes large, it is possible to decide an assist timing more accurately.

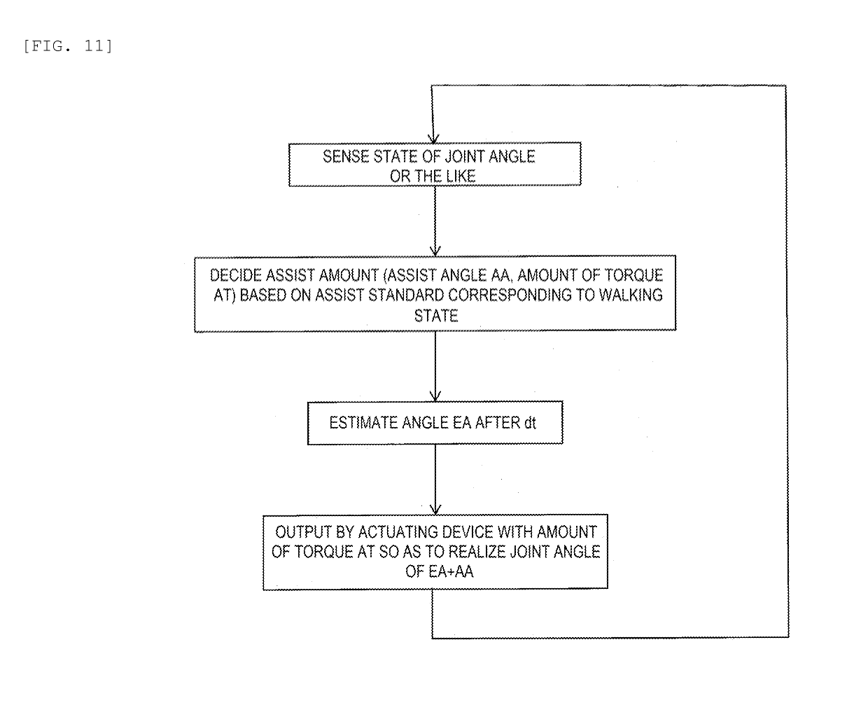

FIG. 11 shows a basic control flow of the wearable power assist system of this example. Signals from the angle sensor 18 included in each assist outer skeleton 12 and from the foot pressure measuring sensor 19 included in each measuring insole 13 are converted to the angle and angular velocity of each joint, foot pressure, and change with time in foot pressure, by the operation control unit 15. Next, an assist amount (assist angle AA, amount of torque AT or the like) is decided on the basis of an assist criterion corresponding to a walking state that is set in advance by the operation control unit 15. Next, the angle EA of the joint to be assisted in the time in which the assist outer skeleton is actually operated is predicted. An output amount of the actuating device 16 (the force and amount of pulling the wire 34) is decided in such a way that the joint angle of the assist outer skeleton with the amount of torque AT equals the sum of the assist angle AA and the predicted joint angle EA, and the output is made, thus causing the assist outer skeleton 12 to operate. Such a flow is repeated.

The assist criterion corresponding to the walking state is a criterion that defines which assist should be given in which walking state. This varies depending on the user's walking habit and the requested assistance and therefore needs to be set in advance suitably for the user.

As an example of a setting method, a knee assist setting method for reducing fatigue at the time of stair ascent will be described. Here, a setting method based on knee angular velocity and information of sole pressure will be described.

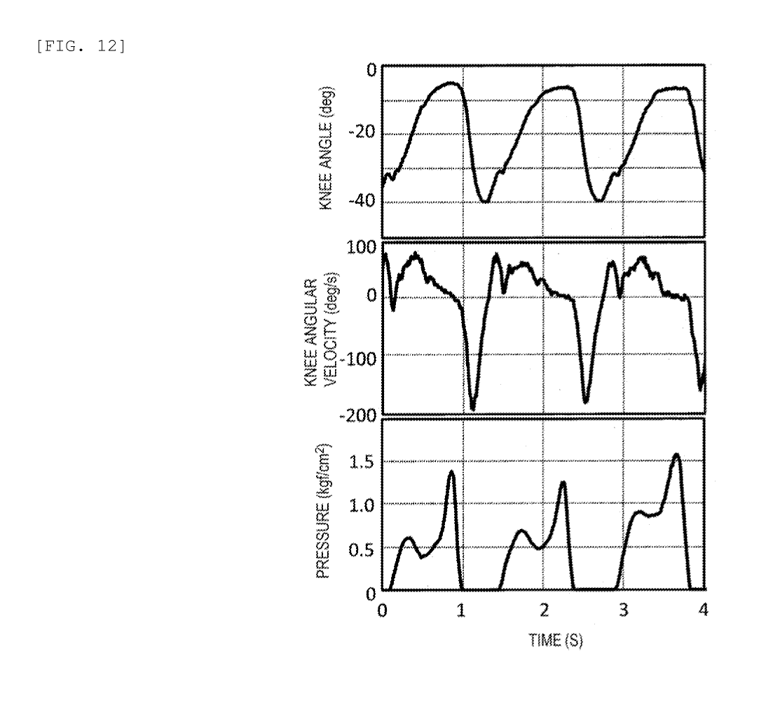

FIG. 12 shows a typical example of change with time in the knee joint angle of the right leg, the speed of the knee joint angle, and the sole pressure at the time of stair ascent. In this illustration, the knee angle is 0 degrees when fully extended, and the bending direction is referred to as the negative direction. The major part of fatigue at the time of stair ascent is due to the act of lifting up the body one step above against gravity. Therefore, in this case, assistance with the knee joint is carried out by the assist outer skeleton, when the body weight is placed on the foot and the knee joint shifts from a bent state to an extended state. That is, assistance is carried out when the knee angular velocity is v1 or above, which is a positive value, and the sole pressure is p1 or above. As for the absolute values of v1 and p1, an amount that the user feels comfortable is actually measured and set because the comfortable timing varies from user to user. The assist operation at this time is to extend the knee joint. However, operating the assist outer skeleton at the same speed to the same angle as the knee joint does not produce any sense of being assisted. To achieve a sense of being assisted, it is necessary to apply a force with the assist outer skeleton so as to extend the user's knee. This can be realized by performing control such that an angle that is smaller than the actual knee joint angle by dA (slightly extended state) is achieved at the timing of assisting. As for the dA and the torque AT at the time of assisting, an amount that the user feels comfortable is actually measured and set because the comfortable amount varies from user to user. Such an assist operation can also be carried out by controlling the torque applied to the assist outer skeleton.

In other walking states than stair ascent where the user needs assistance, a necessary assist operation, a condition for assist timing, and an assist amount are similarly found on the basis of the joint angle information and the sole pressure distribution information of the user, and set as an assist criterion.

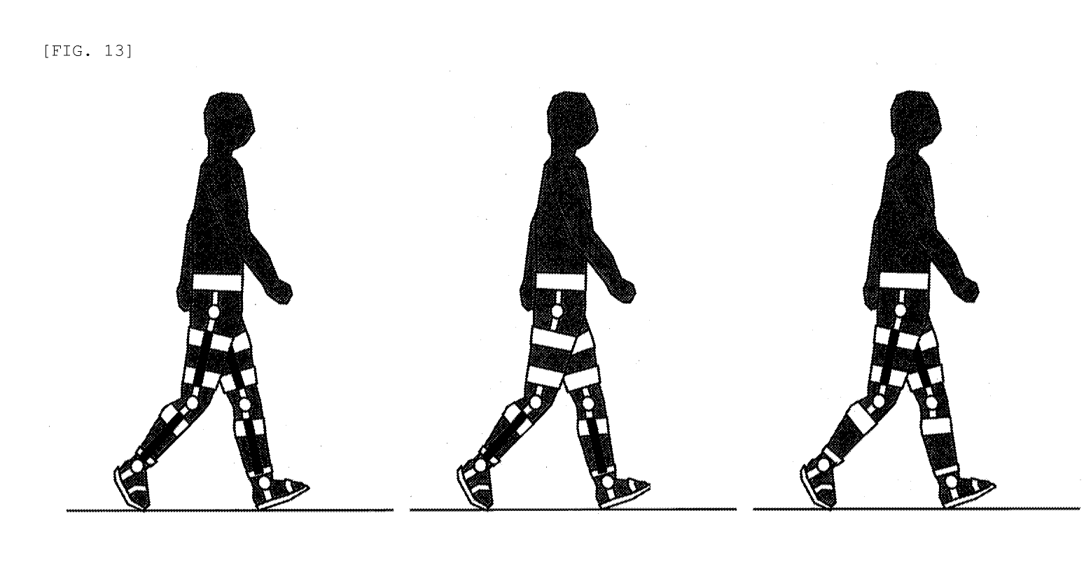

The knee joint assist outer skeleton can be displaced in use more easily than the assist outer skeletons for other joints. Therefore, a structure in which the knee joint assist outer skeleton is jointed to other joint assist outer skeletons, as shown in FIG. 13, enables stable use.

As described above, using the method of this example, a wearable walking assist system that is light-weight and capable of coping with various situations can be provided. In this example, walking assistance is mainly described. However, by changing the joint to be assisted, actions other than walking, such as lifting up a heavy object or moving one's position, can be coped with as in walking assistance.

### EXAMPLE 2

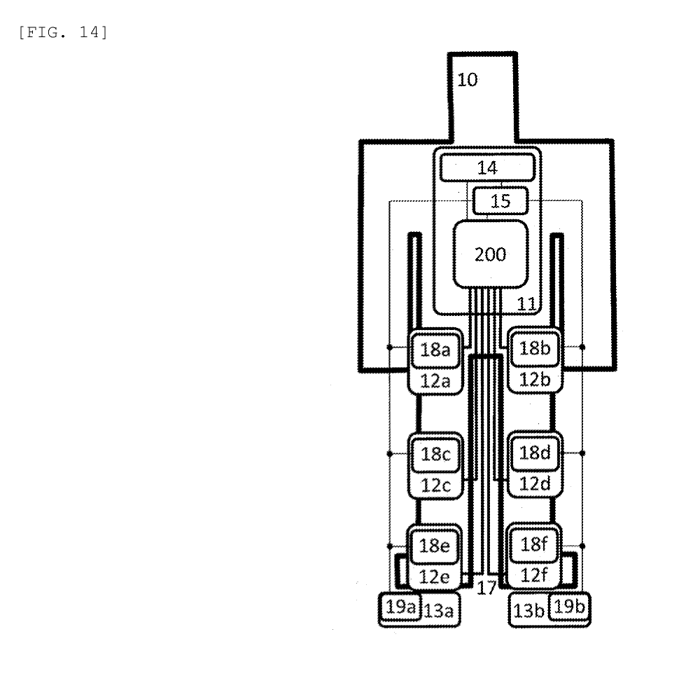

In Example 2, the configuration and operation of an embodiment of the wearable power assist system according to the invention will be described. FIG. 14 is a schematic view for explaining the configuration of a wearable power assist system of this example. The difference between the system of this example and the system of Example 1 is that, in the system of Example 1, the drive force transmission unit 17 is relocated to and used on a required assist outer skeleton, whereas in the system of this example, the drive force transmission unit 17 is joined to all the assist outer skeletons and switched in use by a selective actuating device 200 in such a way that a drive force is transmitted to a required assist outer skeleton.

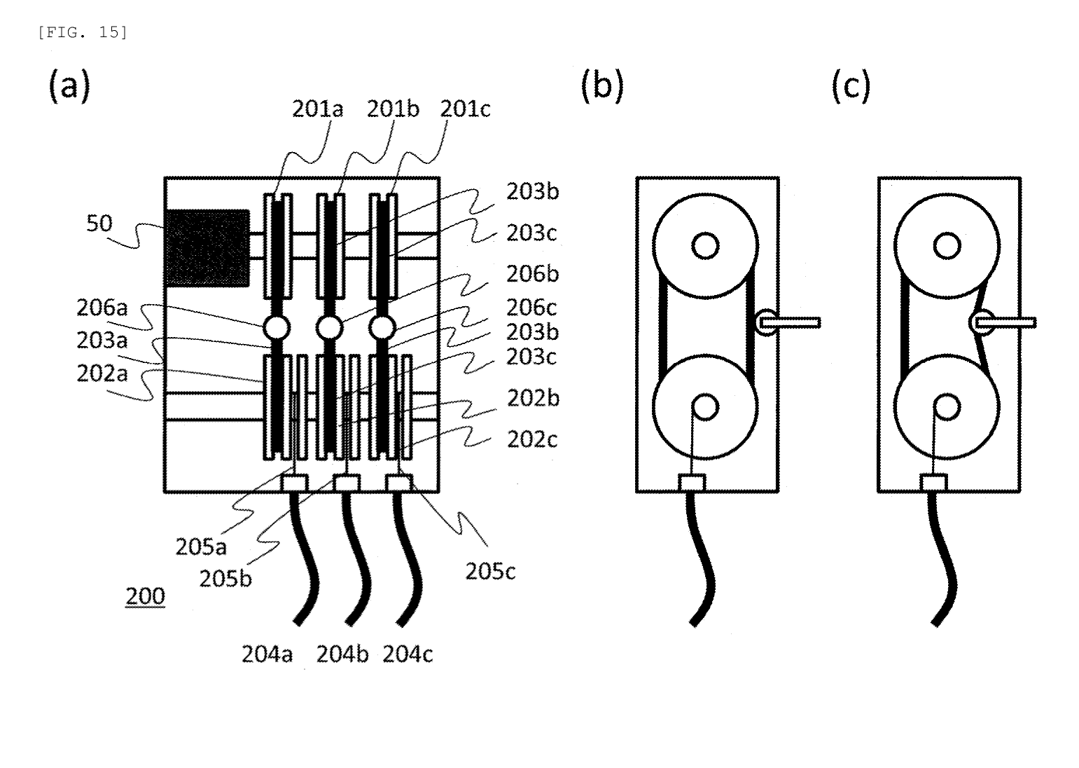

An example of the selective actuating device 200 will be described using FIG. 15. FIG. 15 is a schematic view for explaining actuators and switching devices of the selective actuating device 200. In practice, two sets of actuators and switching devices on the left and right are necessary in the selective actuating device 200. However, here, only one set is illustrated for explanation. FIG. 15(a) is a schematic view seen from the top. FIG. 15(b) is a schematic view seen from the side when a select tension pulley is not pressed. FIG. 15(c ) is a schematic view seen from the side when a select tension pulley is pressed. Belts 203a, 203b, 203c are laid on three motor pulleys 201a, 201b, 201c and operation pulleys 202a, 202b, 202c. The motor pulleys 201a, 201b, 201c are fixed to the rotating shaft of a motor 50 and rotate simultaneously as the motor rotates. The operation pulleys 202a, 202b, 202c rotate independently of each other. Moreover, wires 205a, 205b, 205c of drive force transmission units 204a, 204b, 204c are connected to the operation pulleys 202a, 202b, 202c. Although not shown, the drive force transmission units 204a, 204b, 204c are connected to the hip joint assist outer skeleton, the knee joint assist outer skeleton, and the ankle joint assist outer skeleton, respectively. There are select tension pulleys 206a, 206b, 206c near the belts 203a, 203b, 203c. As one of these is selected and pressed, the rotating force of the motor is transmitted to the operation pulley to which the belt with the select tension pulley pressed thereon is linked, and the assist outer skeleton connected thereto can be operated. The motive power switching mechanism in the selective actuating device can be a switching mechanism that is similar to a bicycle derailleur, other than the mechanism described with reference to FIG. 15.

For the motive power switching in the selective actuating device, it is possible to estimate the walking state on the basis of a walking state determination criterion that is set in advance, then determine the joint that needs assistance, and automatically switch the motive power, other than manual operation. The walking state determination criterion is the walking state, the joint angle, and the sole pressure information of the user associated with each other in order to estimate the present walking state. This criterion varies depending on the walking habit of the user and therefore needs to be set in advance suitably for each user.

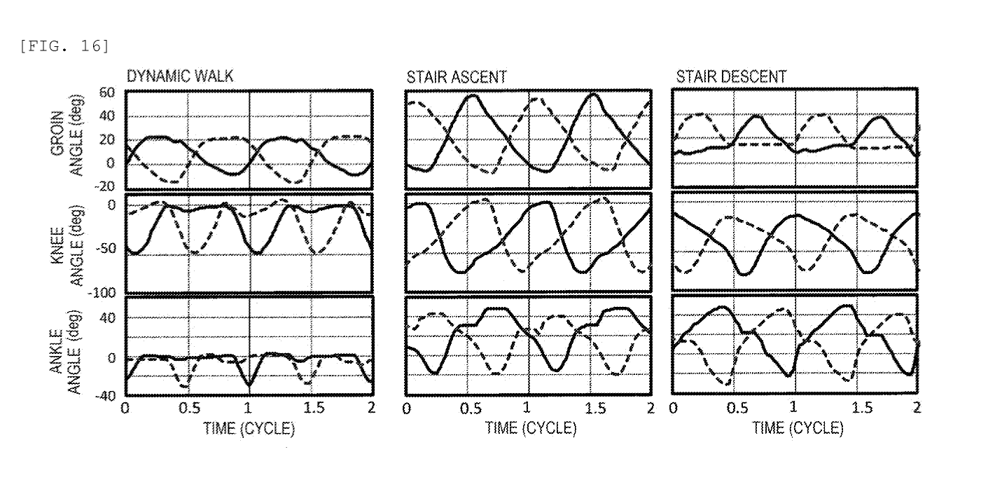

For example, information of change with time in the hip joint angle, the knee joint angle, and the ankle joint angle can be used to determine whether it is flatland walk, stair ascent, or stair descent. FIG. 16 shows a typical example of change with time in each joint in flatland walk, stair ascent, and stair descent. In this illustration, each joint is at 0 degrees when extended. For the hip joint, the direction of lifting forward is the positive direction. For the knee joint, the bending direction is the negative direction. For the ankle joint, the tiptoeing direction is the positive direction. Clearly, it can be seen that information of change with time in the hip joint angle, the knee joint angle, and the ankle joint angle is different among the three walking patterns. Information of change with time in each joint with respect to the walking state where the user needs assistance is measured and recorded in advance. Then, information of change with time in the hip joint angle, the knee joint angle, and the ankle joint angle is measured at the time of using the assist system of this example, and compared with the information of change with time in each joint that is recorded in advance. The closest state is estimated as the walking state at the time.

As described above, using the method of this example, a wearable walking assist system that is light-weight and capable of coping with various situations can be provided. In this example, walking assistance is mainly described. However, by changing the joint to be assisted, actions other than walking, such as lifting up a heavy object or moving one's position, can be coped with as in walking assistance.

### REFERENCE SIGNS LIST

10 . . . user, 11 . . . actuation-control device housing unit, 12 . . . assist outer skeleton, 12a . . . right hip joint assist outer skeleton, 12b . . . left hip joint assist outer skeleton, 12c . . . right knee joint assist outer skeleton, 12d . . . left knee joint assist outer skeleton, 12e . . . right ankle joint assist outer skeleton, 12f . . . left ankle joint assist outer skeleton, 13 . . . measuring insole, 13a . . . right foot measuring insole, 13b . . . left foot measuring insole, 14 . . . power source, 15 . . . operation control unit, 16 . . . actuating device, 17 . . . drive force transmission unit, 18 . . . joint angle sensor, 18a . . . right hip joint angle sensor, 18b . . . left hip joint angle sensor, 18c . . . right knee joint angle sensor, 18d . . . left knee joint angle sensor, 18e . . . right ankle angle sensor, 18f . . . left ankle joint angle sensor, 19 . . . foot pressure measuring sensor, 19a . . . right foot pressure measuring sensor, 19b . . . left foot pressure measuring sensor, 30 . . . motor, 31a . . . right motor, 30b . . . left motor, 31a . . . right pulley, 31b . . . left pulley, 32a . . . wire fixing section, 32b . . . wire fixing section, 32c . . . wire fixing section, 32d . . . wire fixing section, 33 . . . housing pipe, 34 . . . wire, 40 . . . fixed stay, 41 . . . knee pad, 42 . . . power stay, 43 . . . angle sensor, 44 . . . joint angle measuring stay, 45 . . . thigh fixing belt, 46 . . . knee fixing belt, 47 . . . calf fixing belt, 48 . . . power belt, 52 . . . stopper, 53 . . . upper wire guide, 54 . . . lower wire guide, 70 . . . center wire guide, 200 . . . selective actuating device, 201a . . . motor pulley, 201b . . . motor pulley, 201c . . . motor pulley, 202a . . . operation pulley, 202b . . . operation pulley, 202c . . . operation pulley, 203a . . . belt, 203b . . . belt, 203c . . . belt, 204a . . . drive force transmission unit, 204b . . . drive force transmission unit, 204c . . . drive force transmission unit, 205a . . . wire, 205b . . . wire, 205c . . . wire, 206a . . . select tension pulley, 206b select tension pulley, 206c . . . select tension pulley

1. A wearable power assist system comprising:
an actuating device which generates motive power with two actuators;
an operation control unit which controls an operation of the actuating device;
a plurality of assist outer skeletons which includes a pair of outer skeletons mounted on the side of a plurality of joints of a user and having a joint structure, and a fixing member for fixing to the user; and
an action transmission unit which transmits a drive force to the assist outer skeletons from the actuating device;
wherein the assist outer skeletons is made to operate by the actuating device, thereby assisting a movement of the user, and
force generated by one actuator drives the plurality of assist outer skeletons.

2. The wearable power assist system according to claim 1, wherein a force generated by the actuating device is switched and transmitted to the assist outer skeleton that is selected.

3. The wearable power assist system according to claim 2, wherein a force generated by the actuating device is switched and transmitted to the assist outer skeleton that selects the action transmission unit.

4. The wearable power assist system according to claim 2, wherein the assist outer skeleton that is necessary is selected according to an action state of the user, and a force generated by the actuating device is automatically switched and transmitted thereto.

5. The wearable power assist system according to claim 2, wherein an action of the user is estimated from a joint angle of the user, and the assist outer skeleton is made to carry out an assist action corresponding to the estimated action of the user from among actions that are set in advance.

6. The wearable power assist system according to claim 5, wherein an action of the user is estimated from pressure distribution applied to a sole of the user, and the assist outer skeleton is made to carry out an assist action corresponding to the estimated action of the user from among actions that are set in advance.
